(19) United States
(12) Patent Application Publication
(10) Pub. No.: US 2006/0013889 A1
(43) Pub. Date: Jan. 19, 2006

(54) Colostrum-based treatment for irritable bowel syndrome

(76) Inventors: Raymond J. Playford, London (GB); Randall P. Kjelden, Hendricks, MN (US)

Correspondence Address: MERCHANT & GOULD PC, P.O. BOX 2903, MINNEAPOLIS, MN, 55402-0903, US

(21) Appl. No.: 10/892,939

(22) Filed: Jul. 16, 2004

(51) Int. Cl. A61K 35/20; A61K 38/40

(52) U.S. Cl. 424/535; 514/2

(57) ABSTRACT

A method for prophylactically treating irritable bowel syndrome in a mammal through the administration of a colostral composition like bovine colostrum is provided, although other forms of colostrum or other components exhibiting colostrum activity like lactoferrin, casein, or whey may be used. The effective amount of colostral composition to be used will depend upon such factors as the age and weight of the mammal, the bioactivity level of the colostral composition, and whether treatment of existing IBS symptoms, or prevention of the onset of IBS symptoms is desired. A medicament comprising such a colostral composition for prophylactically treating IBS in a mammal is also provided.

### FIELD OF THE INVENTION

This invention relates to a method for using a colostral composition to prophylactically treat irritable bowel syndrome in mammals, and colostral compositions for such treatments.

### BACKGROUND OF THE INVENTION

Irritable bowel syndrome (“IBS”), also known as “spastic colon,” “mucous colitis,” “spastic colitis,” “nervous stomach,” or “irritable colon,” is a functional disorder of the digestive system in which the small and large intestine do not work properly. While IBS is not a disease, it can cause a number of painful or otherwise unpleasant symptoms in the abdominal area like cramping, bloating, gas, diarrhea, and constipation. At any given time, 10-20% of the general population suffers from IBS, with woman under age 45 being particularly afflicted.

In the human or animal body, food is passed from the stomach to the duodenum portion of the small intestines where it is digested, and then to the jejunum and ileum portions of the small intestines where the nutrients (e.g., fatty acids, sugars, amino acids) from the digested food are absorbed. The remaining digesta is then passed along to the large intestine (sometimes called the “bowel” or “gut”) in which fluids from this solid mass of digested food are absorbed. Finally, the leftover, unused portion of the food enters the rectum for subsequent discharge from the body through the anus.

In terms of effect, IBS seems to render the nerves and muscles within the small and large intestine extra-sensitive. For instance, the muscles may contract too much when the person or animal eats, thereby resulting in cramping or diarrhea during or shortly after the meal. The nerves can also be overly sensitive to the stretching of the large intestine as the digesta passes through it due, for example, to gas. Cramping or pain can result.

The prevalence of IBS within society is demonstrated by the British Society of Gastroenterology Guidelines for the Management of Irritable Bowel Syndrome that comprehensively describes the symptoms, diagnosis, and treatment for this functional disorder. The counterparts within the United States are the American College of Gastroenterology and the American Gastroenterology Association. Besides abdominal pain or discomfort, IBS can result in other non-gastroenterological disorders like lethargy, poor sleep, fibromyolgia, backache, urinary frequency, and dyspareunia. IBS can also lead to substantial reductions in quality of life, including refusals of social invitations, and restrictions upon travel and work. Patients in the U.S. suffering from IBS lost an average of 14.8 workdays per year, compared to 8.7 workdays for the asymptomatic population. See Drossman, D. A., Li, Z., Andruzzi, E., et al. “U.S. Householder Survey of Functional Gastrointestinal Disorders. Prevalence, Sociodemography, and Health Impact”, Dig. Dis. Sci 38: 1569-80 (1993). IBS patients also exhibit greater tendencies for anxiety, hostile feelings, sadness, depression, interpersonal sensitivity, and sleep disturbances when compared with the population as a whole. See Whitehead, W. E., Engel, B. T., and Schuster, M. M., “Irritable Bowel Syndrome Physiological and Psychological Differences Between Diarrhea-Predominant and Constipation Predominant Patients”, Dig. Dis. Sci. 20: 404-13 (1980); Svedlund, J., Sjodin, I., Dotevall, G., et al., “Upper Gastrointestinal and Mental Symptoms in the Irritable Bowel Syndrome,” Scand. J. Gastroenterol., 20: 595-601 (1985); Gamborone, J., Dewsnap, P., Libby, G., et al., “Abnormal Illness Attitudes in Patients with Irritable Bowel Syndrome,” J. Psychosom. Res. 39: 227-30 (1995); Ford, M. J., Miller, P. C., Eastwood, J., et al. “Life Events, Psychiatric Illness, and the Irritable Bowel Syndrome,” Gut 28: 160-65 (1987).

While IBS and inflammatory bowel disease (“IBD”) are sometimes confused by the general public because of their similar names, and because they both have effects upon the bowel, these conditions are, in fact, completely distinct in terms of appearance, treatment, and future prognosis. For instance, the main underlying problem caused by IBS is increased sensitivity of the bowel nerves, while IBD produces gross inflammation that is out of control. The main symptoms of IBS, as described above, are bloating and changes in bowel habits, while the symptoms of IBD are passing blood and mucus, and weight loss. The visual appearance of the intestinal lining of a person suffering from IBS is normal, while the intestinal lining of a person suffering from IBD bleeds and is ulcerated. At a microscopic level, the intestinal lining will appear essentially normal in the case of IBS, while lots of inflammatory cells will be present in the case of IBD. For IBS, the symptoms may come and go, and are treatable by changes in diet and administration of drugs. In the case of IBD, if drugs do not eliminate the symptoms, surgery may be required.

Some people exhibit symptoms of IBS in their youth, while others do not develop symptoms until later in life. Young adult women most typically develop IBS symptoms. This may suggest that infection is a cause of IBS. Indeed, a recent study has demonstrated that patients who suffered from bacterial gastroenteritis are ten times more likely to develop IBS one year later compared with the general population. See Rodriguez, L. A. G. and Ruigomez, A., “Increased Risk of Irritable Bowel Syndrome After Bacterial Gastroenteritis: Cohort Study,” B. M. J. 318: 565-66 (1999). Moreover, although IBS has generally been regarded as a condition where no inflammation is occurring, as opposed to predominantly inflammatory diseases in the small and large intestine such as IBD, Crohn's Disease, and ulcerative colitis, more detailed studies suggest that inflammation may be occurring at the microscopic level in the small and large intestine with increased numbers of lymphocytes. See Tomblom, H., Lindberg, G., Nyberg, B., and Veress, B., “Full-Thickness Biopsy of the Jejunum Reveals Inflammation and Enteric Neuropathy in Irritable Bowel Syndrome,” Gastroenterology 123: 1972-79 (2002). It has also recently been shown that resetting of the nerves in the intestinal wall may occur during intestinal inflammation, particularly when caused by infection. See Stead, R. H., “Nerve Remodeling During Intestinal Inflammation,” Ann. N.Y. Acad. Sci. 664: 443-55 (1992).

Commonly accepted treatment of IBS focuses upon diet changes, medicine, and stress relief. Some foods that have been found to cause IBS symptoms are fatty foods like french fries, milk products like cheese or ice cream, chocolate, alcohol, caffeine found in coffee, tea, and some sodas, and carbonated drinks. Thus, a doctor may advise an IBS patient to reduce or cease altogether the consumption of these food items. At the same time, increased consumption of fiber-intense foods like bran, bread, cereal, beans, fruit, and vegetables can make stools softer, bulkier, and easier to pass, thereby relieving constipation. IBS patients suffering from diarrhea, by contrast will often be advised to reduce fiber consumption.

Dietary change, by itself, however, will usually be insufficient to treat IBS. Therefore, doctors often prescribe medicines like laxatives to treat constipation, antispasmodics to slow contractions in the large intestine to help with diarrhea and pain, and antidepressants to help those IBS patients suffering from severe pain. Powerful steroid drugs, often used for inflammatory bowel conditions such as Crohns disease and ulcerative colitis, are ineffective for patients with irritable bowel syndrome. This was also shown in a recent study where administration of predinisolone was found to be ineffective for treating post-infectious IBS. See Dunlap, S. P., Jenkins, D., Neal, K. R., et al., “Randomized, Double-Blind, Placebo-Controlled Trial of Prednisolone in Post-Infectious Irritable Bowel Syndrome,” Ailment Pharmcol. Ther. 18: 77-84 (2003). Moreover, doctors and patients are naturally concerned by long-time use of steroid-based drugs due to the documented side effects. Furthermore, antidepressant medications may be counter-productive for patients with major psychological problems, since their prescription may reinforce abnormal illness behavior and prevent them from effectively addressing underlying psychological problems. Patients can also become dependent upon antispasmodic and antidepressant drugs.

Colostrum is the first milk produced after birth, and is particularly rich in specific anti-microbial factors like immunoglobulins and non-specific antimicrobial factors like lactoferrin. Colostrum also contains a wide variety of immune modulatory “cytokine” type factors, as well as a whole variety of peptide growth factors, such as epidermal growth factor (“EGF”) and insulin-like growth factors I and II. In combination with the milk that is subsequently produced by the mother, colostrum is an important contributor to the nutrition, growth, development, and immunological defenses of the newborn infant.

Bovine colostrum is commonly produced in as a side product by the milk industry. It is currently available in health food stores, where it is usually marketed as a general health-promoting agent. However, it has also been shown that bovine colostrum can be employed to prevent or reduce perforation of the wall of the small intestines caused by non-steroidal anti-inflammatory drugs (“NSAIDs”) like aspirin, ibuprofen, and indamethacin. See Playford, R. J., MacDonald, E., Calnan, D. P., et al., “Co-Administration of the Health Food Supplement, Bovine Colostrum, Reduces the Acute Non-Steroidal Anti-Inflammatory Drug-Induced Increase in Intestinal Permeability,” Clinical Science 100: 627-33 (2001). See also EPO Patent Nos. 927,042 and 936,917 issued to Johnson and Playford. In another study, it has been demonstrated that patients suffering from distal colitis (an IBD) showed marked improvement after taking a colostrum enema in combination with 5-aminosalicylic acid mesalazine, compared with the control group receiving mesalazine and a placebo enema. See Khan, Z., MacDonald, C., Wicks, A. C., et al., “Use of the ‘Nutriceutical’, Bovine Colostrum, for the Treatment of Distal Colitis: Results from an Initial Study,” Ailment Pharmacol. Ther. 16: 1917-22 (2002). For such IBD symptoms, the growth factors contained in the colostrum stimulate the intestinal cells to repair themselves through proliferation and restitution.

However, colostrum therapy has not previously been applied to IBS patients within the medical community, since there is no role for using growth factors to treat the increased sensitivity of the bowel nerves that may cause many of the symptoms of IBS. No scientific or medical tests have shown any efficacy of colostrum for treating IBS. Therefore, the standard treatments for IBS are drug-based.

### SUMMARY OF THE INVENTION

A method for prophylactically treating IBS in a mammal through the administration of a colostral composition is provided according to the invention. The colostral composition preferably is bovine colostrum, although other sources of colostrum, chemically or enzymatically-modified colostrum, colostrum that has been processed to improve or enhance its characteristics or performance, or other components exhibiting colostrum activity like lactoferrin, casein, or whey may be used. The effective amount of colostral composition to be used will depend upon such factors as the age and weight of the mammal, the bioactivity level of the colostral composition, and whether treatment of existing IBS symptoms, or prevention of the onset of IBS symptoms is desired.

A medicament comprising a colostral composition for prophylactically treating IBS in a mammal is also provided according to the invention.

### BRIEF DESCRIPTION OF THE DRAWINGS

FIG. 1 is a bar chart showing the relative levels of thymidine uptake for T cells, dendritic cells, and mixtures thereof, alone and in conjunction with various colostrum solution concentrations.

### DETAILED DESCRIPTION OF THE PREFERRED EMBODIMENT

Use of various colostral compositions to prophylactically treat IBS is provided by the invention. Such invention may take the form of a method for administering an effective amount of such a colostral composition to a patient (be it animal or human), who is suffering from IBS to prophylactically treat the symptoms of such syndrome. Likewise, the invention may take the form of a specific colostral composition that is efficacious for the prophylactic treatment of IBS.

An “effective amount” of a colostral composition is an amount sufficient to prevent, treat, reduce, and/or ameliorate the symptoms and/or underlying causes of IBS. In some instances, an “effective amount” is sufficient to eliminate the symptoms of IBS and, perhaps, overcome IBS, itself. In the context of the present invention, the terms “treat” and “therapy” and the like refer to alleviate, slow the progression, prophylaxis, attenuation, or cure of existing IBS symptoms. “Prevent,” as used herein, refers to putting off, delaying, slowing, inhibiting, or otherwise stopping, reducing, or ameliorating the onset of such IBS symptoms. “Prophylactic treatment,” as used herein, means either the administration of the remedy in the absence of IBS symptoms to prevent the onset or occurrence of IBS symptoms within the large or small intestine, or the treatment of IBS symptoms that already exist within the large or small intestine using the remedy.

Several different criteria for diagnosing IBS have been developed within the medical community, and therefore are readily available for such diagnosis of IBS. The Manning criteria call for abdominal pain relieved by defecation, looser stools with onset of pain, more frequent stools with onset of pain, abdominal distension, passage of mucus in stools, or sensation of incomplete evacuation. The Rome I criteria requires at least three months of recurrent symptoms of: (1) abdominal pain or discomfort relieved by defecation, or associated with a change in stool frequency or stool consistency; and (2) two or more of the following symptoms on at least 25% of occasions or days: altered stool frequency, altered stool form, altered stool passage, passage of mucus, and bloating or distension. The Rome II criteria looks for at least 12 weeks over the past 12 months of abdominal discomfort or pain having two of the following three features: (1) relieved by defecation; (2) associated with a change in frequency of stools; and (3) associated with a change in consistency of stool. A patient satisfying any of these IBS criteria who seeks medical attention will have administered by a doctor a physical examination, and possibly also have additional tests such as blood tests, x-rays of the large intestine, or an endoscopy to support the IBS diagnosis.

For purposes of this invention, the patient to whom the colostral composition is to be administered is preferably a human, but may also include domesticated animals like dogs, cats, and horses, and livestock like pigs and cows.

The colostral composition of the present invention constitutes a component having colostrum activity. This includes: colostrum, itself; chemically or enzymatically-modified colostrum; colostrum that has been processed to change its form or content; colostrum supplemented with one or more additives to improve or enhance its characteristics or performance; ingredients like lactoferrin, casein, or whey that have been extracted from colostrum or milk, or produced from hosts like plants (e.g., tobacco) or bacteria; and food or drink products incorporating such colostrum or other component have colostrum activity for ease of administration to a patient. The colostral composition could also constitute the combination of two or more individual colostral componenets, such as colostrum supplemented with additional lactoferrin.

Colostrum, sometime called “mother's milk”, is a thick yellow fluid produced from the mammary glands during the first hours after birth. This fluid is so important that without it, many newborn mammals would die. It is not only an important source of nutrients such as proteins, carbohydrates, fat, vitamins, and minerals, but it also contains several biologically active molecules that are essential to the body's immune and growth functions.

More specifically, colostrum contains a number of natural components important for health enhancement as follows:
Immune factors that fight viruses, bacteria, yeast, fungus, allergens, and other toxins.
Immunoglobulins in the form of IgG with smaller amounts of IgA, IgD, IgE, and IgM that are protein molecules that are effective in fighting bacterial and viral infections (e.g., colds, flu), parasites and yeast. Immunoglobulins are energizing elements in colostrum that are anti-inflammatory in nature.
Antibodies to more than 19 specific disease-causing pathogens including rotovirus, H. pylori, cryptosporidium, salmonella, candida, streptococcus, staphylococcus, and E. coli.
Proline-rich polypeptides that help regulate the thymus gland to stimulate a weakened immune system, as well as balance an overactive immune system, as is the cause of many autoimmune diseases.
Lactoferrin, an iron-binding protein with antiviral, antibacterial, and anti-inflammatory properties good for the treatment of diseases like cancer, HIV, herpes, chronic fatigue, candida albicans, and other infections.
Glycoproteins that protect the immune and growth factors in colostrum from destruction by the digestive juices in the stomach and intestinal tract.
Lactalbumins that can be highly effective against numerous forms of viruses.
Cytokines like Interleukin 1 & 6, Interferon gamma, and Lymphokines that are involved in cell-to-cell communication, antiviral and anti-tumor activity, and regulation and intensity of immune responses. Cytokines help to increase T-cell activity and stimulate production of immunoglobulins. Interleukin-10 is a potent anti-inflammatory agent that can be a very effective pain reliever, and other interleukins are helpful for fighting cancer.
Lysozymes that help to protect the body from bacterial infections.
Growth factors that stimulate normal growth, as well as help regenerate and accelerate the repair of aged or injured muscle, skin collogen, bone, cartilage, and nerve tissues. They also can stimulate the body to burn fat for fuel instead of the body's own muscle tissue during times of fasting (diet), and build lean muscle. Growth factors can also be used as an effective topical application for burns, injuries, and skin rejuvenation. Such growth factors in colostrum include: epithelial growth factor (“EGF”) for stimulating normal skin growth and repairing cellular tissues; insulin-like growth factor I and II (“IGF-I” and “IGF-II”) for stimulating the repair and growth of DNA and RNA, increasing lean muscle mass, and helping to regulate blood sugar and cholesterol levels; transforming growth factor alpha & beta (“TGF A & B”) for stimulating the proliferation of cells in connective tissue, assisting in the formation of bone and cartilage, and as a therapeutic agent in bone and wound healing—TGF alpha and beta can also help repair tissue, and may support the development of growth of the lining of the intestines; platelet-derived growth factor (“PDGF”) for helping with cell division in connective tissue, smooth muscle, and fibroblasts; and vitamins and minerals that are naturally occurring and naturally balanced.

While colostrum may be sourced from any mammal for purposes of this invention, such as cows, goats, sheep, and buffalo, it preferable is sourced from a cow. The immune and growth factors found in such bovine colostrum are virtually identical to those found in human colostrum, and the immune factors are reportedly four times richer. An additional benefit to bovine colostrum is its special glyco-proteins and protein constituents, which have been shown to be extremely effective in protecting colostrum's active components from the destructive forces of the human body's digestive system.

Bovine colostrum is commercially available from a number of sources. Such manufacturers include Sterling Technology, Inc. of Brookings, S.D.; Immuno-Dynamics of Iowa, Inc. of Perry, Iowa; and Labelle Industries of Ripon, Calif.

The newborn calf requires three quarts of colostrum within the first six hours after birth, and then two quarts within the next twelve hours, to ensure its health and vitality. Therefore, less commercial colostrum for human consumption will be obtainable during this first six-hour time period from the mother cow. After 72 hours, the colostrum starts to become much more like full milk with complete absence or much reduced amounts of the beneficial factors that are important to human health maintenance and restoration. Therefore, the bovine colostrum for purposes of this invention should preferably be collected from the cow within the first 72 hours after birth of the calf, more preferably within the first 48 hours after birth, even more preferably within the first 24 hours after birth, most preferably within the first two milkings following parturition.

It may be beneficial to use colostrum that was obtained from cows that have been certified as free from hormones, pesticides, and antibiotics, so as not to increase other health problems in a person who consumes the colostrum. There are a number of certified, organic dairies in the United States, New Zealand, and other countries that can produce organic colostrum that fits this criteria.

The colostrum of the present invention should also be minimally processed at the lowest temperature possible in order to avoid destruction of its natural components or denaturation of the product. For example, colostrum in liquid form will typically be manufactured as follows: colostrum from the cow will be frozen by the farmer and sent to the colostrum manufacturer. The manufacturer will then thaw the colostrum in order to grade, test, and analyze it for bioactivity and other desired specifications; heat treat the colostrum at approximately 162° F. for 16 seconds; subject the heat-treated colostrum to mechanical separation and removal of the fat component; enzymatic or acid treatment to remove casein; and filtration to yield the final liquid colostrum product. Because such liquid colostrum will be less stable than powdered colostrum, and more subject to spoilage, it will need to be kept at a temperature around 4 degrees centigrade (refrigerator temperature) in order to ensure that it does not spoil.

Alternatively, the colostrum may be spray dried to form a powder, as is known within the industry. U.S. Pat. Nos. 3,956,521 and 4,281,024, both owned by Aktieselskabet Niro Atomizer, describe processes and equipment for spray drying milk products to produce powders, and are incorporated herein by reference. In order to produce such powdered colostrum, colostrum from the cow will typically be processed as described above through the heat-treatment step. It will then be cooled down and then spray dried. The resulting colostrum powder will be a stable product that can be stored under normal ambient conditions without spoiling or degradation of the vital components in the colostrum, although it may also be defatted to provide additional storage stability. A principal advantage of powderized colostrum is that it may be conveniently contained in a capsule or tablet using technology that is readily known in the art. Alternatively, the colostrum may be purchased as a bulk powder that can be added in a measured amount to water, a drink, or food item for easy consumption. Examples of such drinks include milkshakes, protein shakes, drink concentrates, and fruit juices. Examples of such food items include energy bars, candy bars, yoghurt, kefir, cottage cheese, soft cheese, and ice cream. Liquid colostrum may be drunk by itself in a measured amount by the patient, or added in a measured amount to another drink or food item, such as the ones listed above.

Finally, it is envisioned under this invention that the colostral composition may be prepackaged in a measured amount in a drink or food item, so as to administer for convenience the correct dosage of the colostral composition to a patient for prophylactically treating IBS. The possible list of prepackaged drink or food items includes, but is not limited to, those mentioned above.

One or more additives may advantageously be added to the colostral composition of the present invention to improve or enhance its characteristics or performance. For example, because colostrum can have a natural off flavor, a natural or artificial form of a flavoring agent like licorice, cherry, orange, chocolate, strawberry, vanilla, butter, or lemon can be added to the liquid or powdered colostrum product to make it more palatable to swallow. Approximately 1 ml of the flavoring agent per 5 ounces of liquid colostral composition or 200 g of colostral composition powder could be used. Licorice has the added benefit of changing the contractions of the intestines that can otherwise lead to diarrhea or constipation, and reducing the amount of acid produced in the stomach.

Colostrum can also be straw-colored, which can look unappealing. Thus, a coloring agent can be added to the liquid or powdered colostrum product in order to encourage a person to consume it. A number of natural and artificial colorants are well known within the food industry, and therefore available to the practioner of this invention. Coloring agents may also be beneficial to gelatin capsules containing colostrum.

Soy flour or another soy product like soy protein isolate may also be added as an ingredient to the liquid or powdered colostral composition using techniques known in the industry. Such soy flour or other soy product like soy protein isolate will protect the colostral composition from being digested in the stomach and the first section of the small intestine before it can provide the medical benefit to the later parts of the small and large intestine that are involved in causing the symptoms of the IBS. Soy flour can be added in the range of about 15-25% by weight to the end product containing the colostral composition. Likewise, soy protein isolate can be added in the range of about 5-15% by weight to the end product containing the colostral composition.

This invention also includes the addition to the colostral composition of other additives like probiotics to enhance microflora in the intestine.

For purposes of this invention, the colostral composition may be administered to a patient as a prophylactic treatment for IBS in any of the forms discussed above. In the case of a human age 16 or above averaging 70 kg in weight, liquid colostral composition should be administered in a single or multiple daily doses amounting in total to 5-200 ml/day, more preferably 15-80 ml/day, even more preferably 15-30 ml/day. If administered in powdered form, the single or multiple daily dosage of colostral composition should total 1-100 g/day, more preferably 3-20 g/day, even more preferably 10-15 g/day.

The colostral composition may also be effective for preventing the onset or reducing the incidence of IBS when taken on a regular basis over time. This could be a daily, in one or divided doses or taken on an intermittent basis. For example, a human age 16 or above averaging 70 kg in weight who is not suffering yet from IBS symptoms should take single or multiple doses amounting in total to 2-70 ml/day, more preferably 10-30 ml/day, even more preferably 10-15 ml/day, as a preventive treatment against the onset of IBS symptoms. The corresponding total colostral composition dosage on a powder basis would be 330 mg-33 g g/day, more preferably 1-7 g/day, even more preferably 2-4 g/day.

For children younger than age 16, the dosages discussed above for treating or preventing IBS should be cut in half. The dosages may also be adjusted to take into account the weight of the patient, as is well known in the medical arts. This is particularly important for administering the colostral composition to patients that are not humans.

In order to ensure the effectiveness of the colostral composition medicament of the present invention for the treatment of IBS, it is important to determine that the colostral composition product has a sufficient level of biological activity using measurements of cell growth and cell movement. Not all colostral composition products, or even samples of the same colostral composition product, exhibit the same level of biological activity because bioactivity of a colostral composition is affected by its preparation, storage, and manufacturing practices. Therefore, it is insufficient merely to focus upon total protein and immunoglobulin levels as is typically done within the colostrum industry. It is well known in the art to employ a “proliferation” assay using intestinal cells and thymidine incorporation to measure cell growth. Moreover, it is well known in the art that a “restitution” assay using intestinal cells where a standard wound has been induced followed by determination of how quickly the cell gap around the wound closes may be employed to measure cell movement. See Playford, R. J., Floyd, D. N., MacDonald, C. E., et al., “Bovine Colostrum Is a Health Food Supplement Which Prevents NSAID Induced Gut Damage,” Gut 44: 653-58 (1999). It is believed for purposes of this invention that the colostral composition constituent should have sufficient activity such that its ability to stimulate growth (proliferation) of human intestinal cells in a culture medium at a final concentration of 1 mg protein/ml, is at least 40% above the baseline level of growth for a corresponding intestinal cell in a culture medium without the colostral composition or any other pro stimulant. Furthermore, its ability to stimulate movement (restitution) of these intestinal cells in the culture medium at a final concentration of 1 mg protein/ml should be at least 40% above the baseline level of movement for a corresponding intestinal cell in a culture medium without the colostral composition or any other pro stimulant. When either or both of these activities are present within the colostral composition, we consider this will provide a sufficient level of bioactivity for the prophylactic treatment of IBS at an appropriate dosage.

While not wanting to be bound by any particular theory, it is plausible that one reason why patients develop IBS may be that an infection within the small or large intestine causes continuing microscopic inflammation that results in changes in the enteric nervous system, which, in turn, increases the sensitivity of the small or large intestine. Factors that have immune-modulatory activity may, therefore, influence the natural history and severity of IBS acting through pathways such as the enteric nervous system, as well as gut flora. Thus, while it should be clear that there is no role for stimulated growth factors in treating IBS, the inventor wanted to determine whether immune factors in colostral compositions like colostrum might be effective in dampening down the inflammation of the intestinal wall, thereby reducing, influencing, and/or resetting the nerves in the intestinal walls that appear to be important in the causation of IBS symptoms. Therefore, the following experiment was conducted to test this theory.

### Experiment

Human stimulated monocyte-derived dendritic cells (also known as “D cells”) were isolated by using CD14-MACS beads, and grown in X-VIVO 15 medium, containing 10% human serum, 50 mg/ml interleukin 4, and granulocyte-macrophage-colony stimulating factor for 6 days. The dendritic cells were also incubated with 100 mg/ml lipopolysaccheride for 24 hours in order to increase (also known as “active” or “mature”) their responsiveness.

The first set of dendritic cells were plated out in a round-bottomed 96-well plate at 10,000 cells per well in 100 ml medium either alone or in combination with another person's T cells. The second group of responding cells were non-adherent peripheral blood monocytes (T cells) obtained from a different donor. The T cells were also plated out at 100,000 cells per well either alone or in combination with the dendritic cells.

In some wells, bovine colostrum obtained from Sterling Technology was added at 1%, 5%, and 10% concentrations. For instance, 20 ml of colostrum in a 200 ml total volume equated to a 10% colostrum solution. The plates were then incubated for 4 days to allow the cells to react together. In order to analyze this reaction, on Day 4, 1 μCi of tritiated radioactive thymidine was added to each well, and the plates were then incubated for a further 16-18 hours.

The plates were harvested onto glass filter mats, and read using liquid scintillation spectroscopy in a beta counter. The results were expressed as mean counts per minute (cpm) for triplicate cultures. Error bars are standard deviations from the mean.

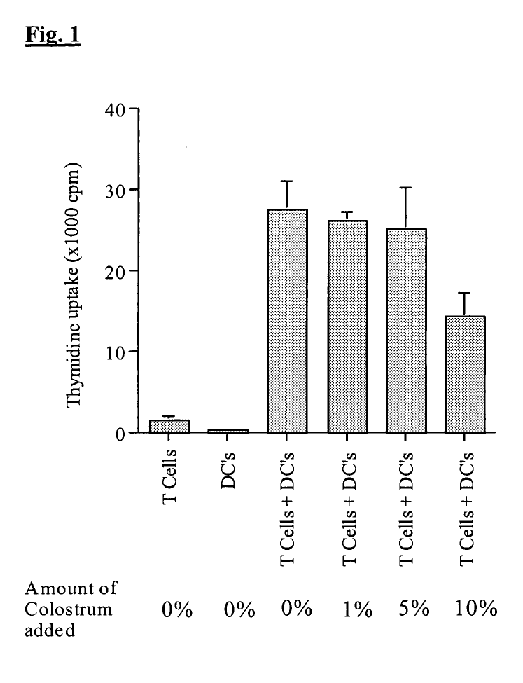

The experimental results are shown in FIG. 1. Ordinarily, the more that the cells fight each other, the greater increase in cell numbers that will result. DNA will be manufactured pursuant to this increase in cell numbers. Radioactive thymidine was therefore used as a marker to measure how many cells were present in each culture.

The higher the value on the Y-axis of FIG. 1, the more that the cells have reacted to each other. T cells and dendritic cells do not fight with themselves, so one would not expect very much growth in their number under the experiment, which is confirmed by the first two bars of the chart in FIG. 1. One would expect, however, that a mixture of T cells and dendritic cells would fight each other, which, indeed, is evidenced by the 28,000 cpm value for thymidine uptake by the third bar in FIG. 1.

If T cells and dendritic cells in the intestine are, in fact, fighting against the nerves to make them more sensitive and cause inflammation, thereby resulting in the symptoms of IBS, then one would want to reduce the number of cells fighting against each other in order to diminish this inflammatory response. As can be seen from FIG. 1, addition of a 1% colostrum solution to the T cell/dendritic cell mixture caused a small but determinable reduction in cells to approximately 26,000 cpm. A 5% colostrum solution caused a greater reduction in overall number of cells in the T cell/dendritic cell mixture to 25,000 cpm. A 10% colostrum solution on the other hand caused a significant reduction in overall cell numbers to 13,000 cpm.

Thus, these experimental results appear to confirm that colostrum can be used to achieve an immune response in the intestine that will dampen down the excessive response caused by the inflammatory cells that are fighting against each other and against the nerves in the intestinal walls, thereby reducing the local microscopic inflammation. A good “dose response” is shown by the results in FIG. 1—i.e., the more colostrum that is added to the T cell/dendritic cell mixture, the greater the dampening effect on the immune response that occurred. This result is in keeping with an immune modulatory effect on cells relevant to the aetiology of IBS. Therefore, this suggests that colostrum can be successfully used to prophylactically treat IBS.

The successful use of colostrum to treat IBS is surprising, because, since inflammation, abnormal nervous innervations, and interactions between the two have not generally been recognized as part of the pathophysiology of the IBS functional disorder, colostrum has not previously been deemed to be a relevant or appropriate treatment or preventive remedy for IBS within the medical community. Moreover, it would be counterintuitive to administer a milk-derived product like colostrum to treat IBS, which is exacerbated by milk products in many patients with IBS.

At the same time, colostrum is a natural product which, based on current evidence, appears to have little or no side effects. It therefore can be taken long-term as a preventive agent, unlike many of the drug treatments currently used for IBS. Because of the possibility that factors like casein in the colostrum may slow gut transit, this novel approach may have particular benefit for patients suffering from the diarrhea-predominant form of IBS.

The above specification, examples and data provide a complete description of the invention relating to the method of prophylactic treatment of IBS, and the composition for such prophylactic treatment. Since many embodiments of the invention can be made without departing from the spirit and scope of the invention, the invention resides in the claims hereinafter appended.

We claim:

1. A method of using a medicament comprising a colostral composition for the prophylactic treatment of irritable bowel syndrome in a mammal.

2. The method according to claim 1, wherein the colostral composition comprises colostrum.

3. The method according to claim 2, wherein the colostrum is obtained from a cow.

4. The method according to claim 1, wherein the colostral composition comprises lactoferrin, casein, or whey that has been extracted from colostrum or milk, or produced from plants or bacteria.

5. The method according to claim 1, wherein the colostral composition has sufficient bioactivity, wherein when it is added to an intestinal cell in a culture medium at a final concentration of 1 mg protein/ml, its ability to stimulate growth (proliferation) of intestinal cells is at least 40% above the baseline level of growth for a corresponding intestinal cell in a culture medium without the colostral composition or any other pro stimulant, or its ability to stimulate movement (restitution) of such an intestinal cell in a culture medium is at least 40% above the baseline level of movement for a corresponding intestinal cell in a culture medium without the colostral composition or any other pro stimulant.

6. The method according to claim 3, wherein the colostrum is obtained from the cow within the first 72 hours post parturition.

7. The method according to claim 3, wherein the colostrum is obtained from the cow within the first 48 hours post parturition.

8. The method according to claim 3, wherein the colostrum is obtained from the cow within the first 24 hours post parturition.

9. The method according to claim 3, wherein the colostrum is obtained from the cow within the first two milkings following parturition.

10. The method according to claim 1, wherein the colostral composition comprises spray-dried colostrum.

11. The method according to claim 10, wherein 330 mg-100 g of spray-dried colostrum is to be administered daily.

12. The method according to claim 11, wherein 1-100 g of spray-dried colostrum is to be administered daily for the treatment of the mammal suffering from IBS symptoms.

13. The method according to claim 11, wherein 330 mg-33 g of spray-dried colostrum is to be administered daily for the prevention of the onset of IBS symptoms in the mammal.

14. The method according to claim 1, wherein the colostral composition comprises colostrum is in liquid form.

15. The method according to claim 14, wherein 2-200 ml of liquid colostrum is to be administered daily.

16. The method according to claim 15, wherein 5-200 ml of liquid colostrum is to be administered daily for the treatment of the mammal suffering from IBS symptoms.

17. The method according to claim 15, wherein 2-70 ml of liquid colostrum is to be administered daily for the prevention of the onset of IBS symptoms in the mammal.

18. The method according to claim 1, wherein the colostrum composition is administered by an oral route.

19. The method according to claim 18, wherein the colostral composition is administered in the form of a capsule, tablet, liquid, food product, or drink product.

20. The method according to claim 1, wherein the medicament further comprises a flavoring agent.

21. The method according to claim 20, wherein the flavoring agent is licorice.

22. The method according to claim 1, wherein the medicament further comprises a coloring agent.

23. The method according to claim 1, wherein the medicament further comprises soy flour or soy protein isolate.

24. The method according to claim 23, wherein the soy flour comprises between about 15-25% of the medicament by weight.

25. The method according to claim 23, wherein the soy protein isolate comprises between about 5-15% of the medicament by weight.

26. A method of prophylactically treating irritable bowel syndrome in a mammal comprising ingesting a colostral composition in an amount effective to reduce the symptoms of IBS, or prevent the onset of the IBS symptoms.

27. The method according to claim 26, wherein the colostral composition comprises colostrum.

28. The method according to claim 27, wherein the colostrum is obtained from a cow.

29. The method according to claim 26, wherein the colostral composition comprises lactoferrin, casein, or whey that has been extracted from colostrum or milk, or produced from plants or bacteria.

30. The method according to claim 26, wherein the colostral composition has sufficient bioactivity, wherein when it is added to an intestinal cell in a culture medium at a final concentration of 1 mg protein/ml, its ability to stimulate growth (proliferation) of intestinal cells is at least 40% above the baseline level of growth for a corresponding intestinal cell in a culture medium without the colostral composition or any other pro stimulant, or its ability to stimulate movement (restitution) of such an intestinal cell in a culture medium is at least 40% above the baseline level of movement for a corresponding intestinal cell in a culture medium without the colostral composition or any other pro stimulant.

31. The method according to claim 28, wherein the colostrum is obtained from the cow within the first 72 hours post parturition.

32. The method according to claim 28, wherein the colostrum is obtained from the cow within the first 48 hours post parturition.

33. The method according to claim 28, wherein the colostrum is obtained from the cow within the first 24 hours post parturition.

34. The method according to claim 28, wherein the colostrum is obtained from the cow within the first two milkings following parturition.

35. The method according to claim 26, wherein the colostral composition comprises spray-dried colostrum.

36. The method according to claim 35, wherein the effective amount of spray-dried colostrum to be ingested daily is 330 mg-100 g.

37. The method according to claim 36, wherein the effective amount of spray-dried colostrum to be ingested daily for the treatment of the mammal suffering from IBS symptoms is 1-100 g.

38. The method according to claim 36, wherein the effective amount of spray-dried colostrum to be ingested daily for the prevention of the onset of IBS symptoms in the mammal is 330 mg-33 g.

39. The method according to claim 26, wherein the colostral composition comprises colostrum is in liquid form.

40. The method according to claim 39, wherein the effective amount of liquid colostrum to be ingested daily is 2-200 ml.

41. The method according to claim 40, wherein the effective amount of liquid colostrum to be ingested daily for the treatment of the mammal suffering from IBS symptoms is 5-200 ml.

42. The method according to claim 40, wherein the effective amount of liquid colostrum to be ingested daily for the prevention of the onset of IBS symptoms in the mammal is 2-70 ml.

43. The method according to claim 26, wherein the colostrum composition is ingested by an oral route.

44. The method according to claim 43, wherein the colostral composition is ingested in the form of a capsule, tablet, liquid, food product, or drink product.

45. The method according to claim 26, wherein the colostral composition further comprises a flavoring agent.

46. The method according to claim 45, wherein the flavoring agent is licorice.

47. The method according to claim 26, wherein the colostral composition further comprises a coloring agent.

48. The method according to claim 26, wherein the colostral composition further comprises soy flour or soy protein isolate.

49. The method according to claim 48, wherein the soy flour comprises between about 15-25% of the colostral composition end product by weight.

50. The method according to claim 48, wherein the soy protein isolate comprises between about 5-15% of the colostral composition end product by weight.

51. A medicament composition for the prophylactic treatment of irritable bowel syndrome in a mammal comprising a colostral composition.

52. The medicament composition according to claim 51, wherein the colostral composition comprises colostrum.

53. The medicament composition according to claim 52, wherein the colostrum is obtained from a cow.

54. The medicament composition according to claim 51, wherein the colostral composition comprises lactoferrin, casein, or whey that has been extracted from colostrum or milk, or produced from plants or bacteria.

55. The medicament composition according to claim 51, wherein the colostral composition has sufficient bioactivity, wherein when it is added to an intestinal cell in a culture medium at a final concentration of 1 mg protein/ml, its ability to stimulate growth (proliferation) of intestinal cells is at least 40% above the baseline level of growth for a corresponding intestinal cell in a culture medium without the colostral composition or any other pro stimulant, or its ability to stimulate movement (restitution) of such an intestinal cell in a culture medium is at least 40% above the baseline level of movement for a corresponding intestinal cell in a culture medium without the colostral composition or any other pro stimulant.

56. The medicament composition according to claim 53, wherein the colostrum is obtained from the cow within the first 72 hours post parturition.

57. The medicament composition according to claim 53, wherein the colostrum is obtained from the cow within the first 48 hours post parturition.

58. The medicament composition according to claim 53, wherein the colostrum is obtained from the cow within the first 24 hours post parturition.

59. The medicament composition according to claim 53, wherein the colostrum is obtained from the cow within the first two milkings following parturition.

60. The medicament composition according to claim 51, wherein the colostral composition comprises spray-dried colostrum.

61. The medicament composition according to claim 51, wherein the colostral composition comprises colostrum is in liquid form.

62. The medicament composition according to claim 51, wherein the colostral composition further comprises a flavoring agent.

63. The medicament composition according to claim 62, wherein the flavoring agent is licorice.

64. The medicament composition according to claim 51, wherein the colostral composition further comprises a coloring agent.

65. The medicament composition according to claim 51, wherein the colostral composition further comprises soy flour or soy protein isolate.

66. The medicament composition according to claim 65, wherein the soy flour comprises between about 15-25% of the medicament composition by weight.

67. The medicament composition according to claim 65, wherein the soy protein isolate comprises between about 5-15% of the medicament composition by weight.
